(19) United States
(12) Patent Application Publication
(10) Pub. No.: US 2006/0155279 A1
(43) Pub. Date: Jul. 13, 2006

(54) Apparatus and method for concave scoliosis expansion

(75) Inventor: James W. Ogilvie, Brighton, UT (US)

Correspondence Address: KARL R CANNON, PO BOX 1909, SANDY, UT, 84091, US

(73) Assignee: Axial Biotech, Inc.

(21) Appl. No.: 11/259,941

(22) Filed: Oct. 26, 2005

Related U.S. Application Data
(60) Provisional application No. 60/622,999, filed on Oct. 28, 2004.

(51) Int. Cl. A61F 2/30

(52) U.S. Cl. 606/61

(57) ABSTRACT

A device and method for treating scoliosis or other bone conditions. The device may be attached to vertebrae to provide a distraction force on a concave side of a spinal curve to assist in straightening the spine. The device may include receivers for receiving fasteners for attaching the device to the vertebrae. The receivers may allow the fasteners to move a predetermined amount such that constrained movement between the device and the vertebrae may be achieved. The device may include an expander portion between the receivers to create a pushing force. The expander portion may include various different types of biasing mechanisms to provide a damping force as well as to allow the vertebrae to move with respect to each other.

### CROSS-REFERENCE TO RELATED APPLICATIONS

This application claims the benefit of U.S. Provisional Application No. 60/622,999, filed Oct. 28, 2004, which is hereby incorporated by reference herein in its entirety, including but not limited to those portions that specifically appear hereinafter, the incorporation by reference being made with the following exception: In the event that any portion of the above-referenced provisional application is inconsistent with this application, this application supercedes said above-referenced provisional application.

### STATEMENT REGARDING FEDERALLY SPONSORED RESEARCH OR DEVELOPMENT

Not Applicable.

### BACKGROUND

1. The Field of the Invention.

The present disclosure relates generally to methods and devices for treating bones, and more particularly, but not necessarily entirely, to methods and devices for treating scoliosis by expanding a concave side of a spinal curve.

2. Description of Related Art

Some of the current operative methods for treating spinal deformities, particularly scoliosis, include correction of a curve of the spine by some internal fixation device. Some traditional surgical methods of treating scoliosis may include inserting rods along the scoliotic spine to correct the curvature. This method may create problems for the patient due to the inability of the rods to extend as the patient grows. Moreover, the invasive nature of the operative procedure may also cause problems for the patient. The patient may experience discomfort when the rods are implanted as well as continued discomfort while the rods remain in place. Furthermore, because the rods may need to be adjusted after time, multiple invasive surgeries may be required, making the treatment painful and difficult, even to the point of discouraging some patients with scoliosis from seeking treatment.

Fusion of the spine in the corrected state may be accomplished by the placement of bone graft between vertebrae. Fusionless methods of treating spinal deformities are also known involving attaching a tether to vertebrae on the convex curve side of the spine. Deformities may be treated by using the tether to selectively constrain growth in a portion of the convex side of the spine. The tether may include a strand threaded through channels defined in a set of blocks attached to the vertebrae on the convex side of the spine, or spinal staples, often made of a shape memory alloy, attached to vertebrae, the staples spanning the intervertebral disc space. Nonoperative methods also exist for treating spinal deformities and may also be used when applicable.

Despite the advantages of known methods and devices for treating spinal deformities and other bone conditions, improvements are still being sought. The prior art is thus characterized by several disadvantages that are addressed by the present disclosure. The present disclosure minimizes, and in some aspects eliminates, the above-mentioned failures, and other problems, by utilizing the methods and structural features described herein.

The features and advantages of the disclosure will be set forth in the description which follows, and in part will be apparent from the description, or may be learned by the practice of the disclosure without undue experimentation. The features and advantages of the disclosure may be realized and obtained by means of the instruments and combinations particularly pointed out in the appended claims.

### BRIEF DESCRIPTION OF THE DRAWINGS

The features and advantages of the disclosure will become apparent from a consideration of the subsequent detailed description presented in connection with the accompanying drawings in which:

FIG. 1 is a posterior view of a spinal column utilizing a device in accordance with the principles of the present disclosure;

FIG. 2 is a schematic side view of a device distracting vertebrae in accordance with the principles of the present disclosure;

FIG. 3 is a front view of one embodiment of an implant in accordance with the principles of the present disclosure;

FIG. 4 is a side view of one embodiment of a fastener useful with the implant of FIG. 3;

FIG. 5 is a plan view of one embodiment of a catch useful with the fastener of FIG. 4;

FIG. 6 is a side view of an embodiment of an implant and a fastener with a receiver for allowing constrained movement of the fastener;

FIG. 7 is a break-away front view of one embodiment of a receiver useful with a device of the present disclosure;

FIG. 8 is a front view of an alternative embodiment implant;

FIG. 9 is a front view of an additional alternative embodiment implant;

FIG. 10 is a front view of another alternative embodiment implant in an extended position;

FIG. 11 is a front view of the implant of FIG. 10 in a compressed position;

FIG. 12 is a front view of a further alternative embodiment implant;

FIG. 13 is a front view of an additional embodiment implant;

FIG. 14 is a break-away front view of another embodiment of an implant;

FIG. 15 is a front view of one embodiment of a portion of an implant of FIG. 13;

FIG. 16 is a front view of the portion of the implant of FIG. 15 in a compressed condition;

FIG. 17 is a front view of another embodiment of a portion of an implant of FIG. 13;

FIG. 18 is a front view of the portion of the implant of FIG. 17 in a compressed condition;

FIG. 19 is a front view of yet an additional embodiment implant in a contracted position;

FIG. 20 is a front view of the implant of FIG. 19 in an extended position;

FIG. 21 is a front view of another alternative embodiment implant;

FIG. 22 is a break-away view of a portion of a spine including a plurality of implants;

FIG. 23 is a break-away side view of a connection between a plurality of implants;

FIG. 23a is a break-away front view of the connection between a plurality of implants depicted in FIG. 23;

FIG. 24 is a break-away side view of a bone and one embodiment of an implant in accordance with the principles of the present disclosure; and

FIG. 25 is a schematic cross-sectional view of a vertebra showing locations for inserting fasteners.

### DETAILED DESCRIPTION

For the purposes of promoting an understanding of the principles in accordance with the disclosure, reference will now be made to the embodiments illustrated in the drawings and specific language will be used to describe the same. It will nevertheless be understood that no limitation of the scope of the disclosure is thereby intended. Any alterations and further modifications of the inventive features illustrated herein, and any additional applications of the principles of the disclosure as illustrated herein, which would normally occur to one skilled in the relevant art and having possession of this disclosure, are to be considered within the scope of the disclosure claimed.

Before the present devices and methods for treating bones and/or spinal deformities are disclosed and described, it is to be understood that this disclosure is not limited to the particular configurations, process steps, and materials disclosed herein as such configurations, process steps, and materials may vary somewhat. It is also to be understood that the terminology employed herein is used for the purpose of describing particular embodiments only and is not intended to be limiting since the scope of the present disclosure will be limited only by the appended claims and equivalents thereof.

The publications and other reference materials referred to herein to describe the background of the disclosure, and to provide additional detail regarding its practice, are hereby incorporated by reference herein in their entireties, with the following exception: In the event that any portion of said reference materials is inconsistent with this application, this application supercedes said reference materials. The reference materials discussed herein are provided solely for their disclosure prior to the filing date of the present application. Nothing herein is to be construed as a suggestion or admission that the inventors are not entitled to antedate such disclosure by virtue of prior disclosure, or to distinguish the present disclosure from the subject matter disclosed in the reference materials.

It must be noted that, as used in this specification and the appended claims, the singular forms “a,” “an,” and “the” include plural referents unless the context clearly dictates otherwise. Moreover, as used herein, the terms “comprising,” “including,” “containing,” “characterized by,” and grammatical equivalents thereof are inclusive or open-ended terms that do not exclude additional, unrecited elements or method steps.

As used herein, the phrase “dynamic connection” shall be construed broadly to include a connection between two parts in which the parts may be joined together and yet the parts may still be allowed to move with respect to each other.

As used herein, the phrase “constrained movement” shall be construed to include movement of an object with respect to another object in which the movement is limited or inhibited to a predetermined amount of movement in a particular dimension.

As used herein, the term “excursion” shall be construed broadly to include a movement of a part, including a movement outward and back or from a mean position or axis, such as movement allowed by a spring member, as well as motion that may not be oscillating or alternating.

As used herein, the term “distract” shall be construed broadly to include separate or draw, push, or otherwise force one object in a direction away from another, such as when a force is applied to vertebral bodies in a direction that may cause them to separate or reduce the pressure of contact between the bodies, even if the bodies remain in contact.

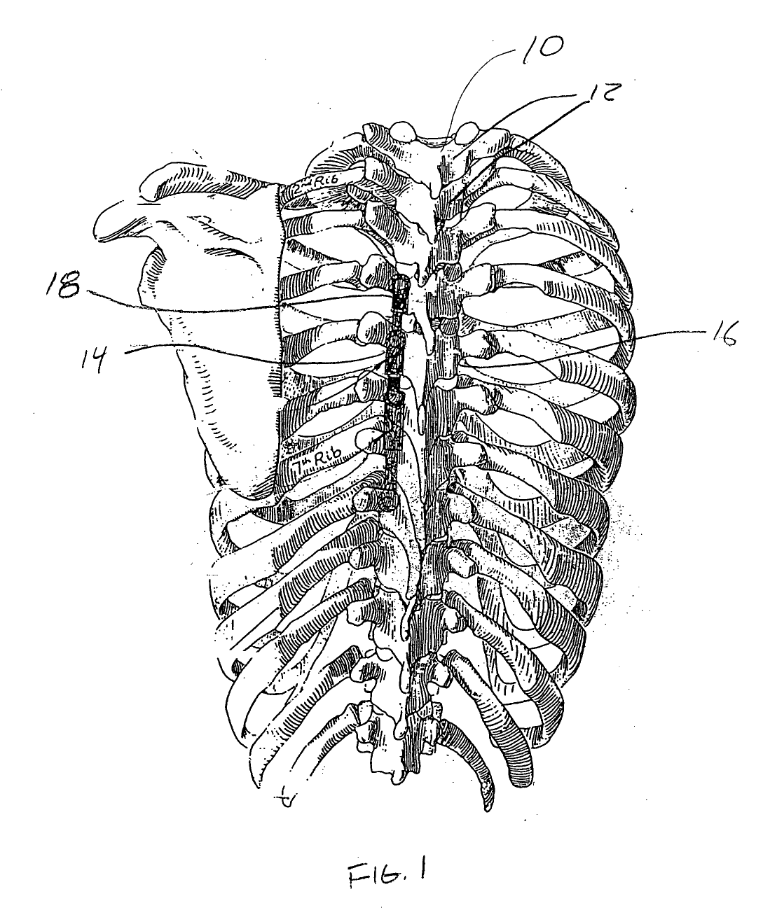

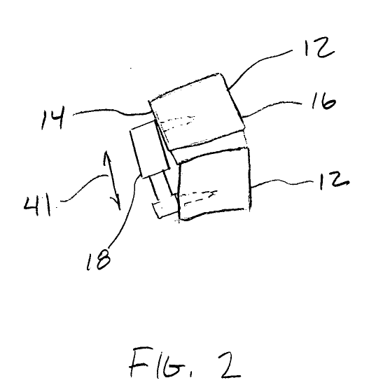

Referring now to FIG. 1, a posterior view is shown of a spine 10 having a plurality of vertebrae 12. The spine 10 may have an abnormal lateral curvature, commonly referred to as scoliosis. The lateral curvature may have a concave side, indicated at 14, and a convex side, indicated at 16, as shown more clearly in FIG. 2. One or more devices or implants 18 may be placed on the concave side 14, to distract or push the vertebrae 12 away from each other to assist in straightening the spine and thereby treat the scoliosis. For example, the Hueter-Volkmann principle states that compressive forces tend to stunt skeletal growth and distractive forces tend to accelerate skeletal growth. Accordingly, a distractive force on the concave side 14 of the spine 10 may tend to accelerate skeletal growth on the concave side 14 to thereby assist in straightening the spine 10. It will also be understood, as discussed more fully below, that the devices and methods disclosed herein may be used to treat other spinal conditions in addition to scoliosis, within the scope of the present disclosure. Moreover, the principles of the present disclosure may be utilized to treat other bone or bone like portions or members not associated with spine.

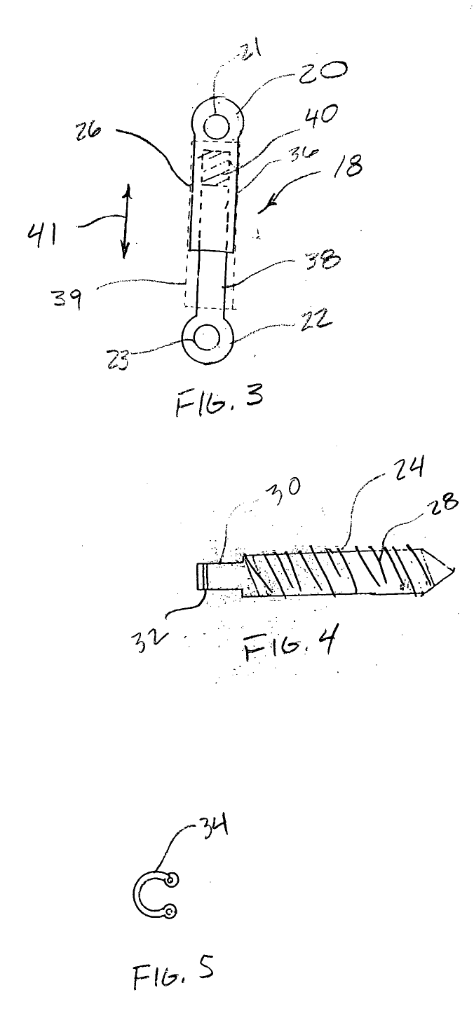

Referring now to FIG. 3, a front view of one embodiment of the implant 18 is shown. The implant 18 may include a first end portion 20 and a second end portion 22. The first end portion 20 may include a first receiver 21 for receiving a fastener 24, as shown most clearly in FIG. 4, to attach the first end portion 20 to a vertebra or other bone or member. The second end portion 22 may include a second receiver 23 for receiving a fastener 24 to attach the second end portion 22 to a vertebra or other bone or member. The first receiver 21 and the second receiver 22 may be configured as openings in the implant 18. Alternatively, the first receiver 21 and the second receiver 22 may have different configurations, such as hooks, or partially spherical members, for example.

The implant 18 may also include an expander portion 26 between the first end portion 20 and the second end portion 22. The expander portion 26 may be configured to provide a distraction force to move the first end portion 20 away from the second end portion 22 in a manner as discussed more fully below. It will be understood that the implant 18 may be sized to be joined to adjacent vertebrae, or the implant 18 may be sized to span multiple vertebrae or other desired span of a bone or bones.

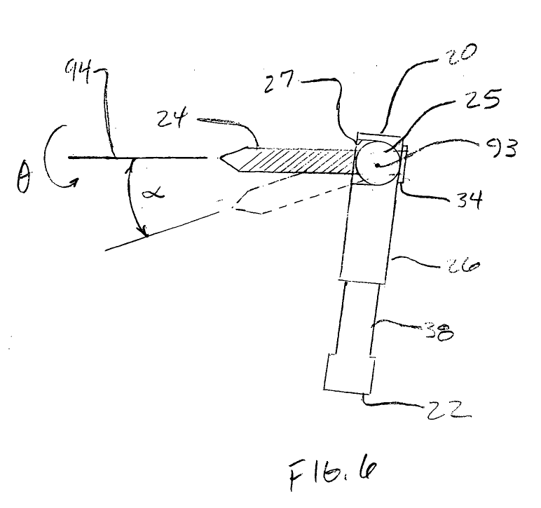

As shown most clearly in FIG. 4, one embodiment of the fastener 24 may include a pedicle screw having threads 28 and a head 30, such that the fastener 24 may be attached to a bone to act as an anchor. The head 30 may have a reduced diameter for being received within the receivers 21, 23. It will be understood that other embodiments of the fastener 24 may include various other suitable types of fasteners, including staples, nails, pins, or screws, for example, including screws having a head with a diameter that may be the same as, or greater than, a diameter of the threaded portion of the fastener. The head 30 may also include a groove 32 for receiving a catch 34, as shown in FIG. 5, for maintaining the head 30 within the receivers 21, 23. One embodiment of the catch 34 may include a “C” ring that may be snapped or pressed into position. Another embodiment of the catch 34 may include a mushroom shaped head that may be threadably engageable with the fastener 24. Moreover, it will be understood that the catch 34 may have various different suitable configurations known to those skilled in the art. Also, other embodiments may include other attaching mechanisms for attaching the fastener 24 to the implant 18.

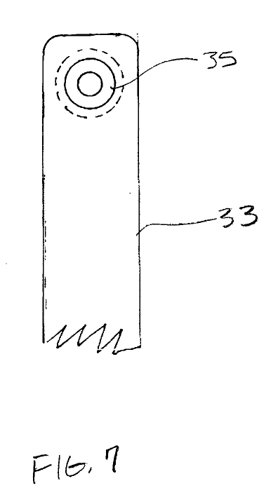

It will be understood that the first receiver 21 and the second receiver 23 may be sized to receive at least a portion of the fastener 24. In one embodiment, at least one of the first receiver 21 and the second receiver 23 may be configured to provide an excursion to allow a predetermined amount of movement of the fastener 24 with respect to the implant 18. For example, as shown most clearly in FIG. 6, one embodiment of the receiver 21, 23 may provide an excursion to allow movement of the fastener 24 through an angle α of up to 20 degrees. Another embodiment of the receiver may be configured to allow movement of the fastener 24 through an angle α of approximately 8 degrees. It will also be understood that the implant 18 may be configured to allow any other suitable movement angle α within the scope of the present disclosure, such as those described more fully in the table below.

Movement of the fastener 24 within a specified angle α may be allowed to provide an excursion to accommodate physiologic growth of the patient, to allow for the natural movement between the vertebrae, and to prevent or reduce the transfer of force that may tend to loosen the fasteners 24 or break the vertebrae or implant 18. Moreover, allowing movement of the fastener 24 may also improve the ease with which surgeons can couple the implant 18 to the vertebrae or other bone portions. Constriction of the movement of the fasteners 24 may prevent the implant 18 from being installed too loosely and may prevent excessive movement of the implant 18 and fasteners 24.

A joint 25, as shown in FIG. 6, such as a convex or rounded member including a movable cylinder, sleeve, spherical bearing, or a bi-polar connection, for example, may be provided within or as part of the receiver 21, 23 to allow movement of the fastener 24 with respect to the implant 18. The joint 25 may be configured to allow movement of the fastener 24 through various ranges of motion such as torsion, flexion and extension, for example. The joint 25 may include an opening for passing the fastener 24 therethrough. It will be understood that any other suitable joint for allowing movement of the fastener 24 with respect to the implant 18 may be used with some embodiments within the scope of the present disclosure, including joints that may be integral with a fastener or joints that may be removable attachable to a fastener.

It will also be understood that the receiver 21, 23 may be configured to constrain the fastener 24 from moving beyond a predetermined point, such that unlimited movement of the fastener 24 may not be possible. For example, the receivers 21, 23 may include a stop 27 for preventing the fastener 24 from moving beyond a particular position. The stop 27 may be formed as a wall or protrusion on the implant 18 or any other suitable mechanism for limiting movement of the fastener 24.

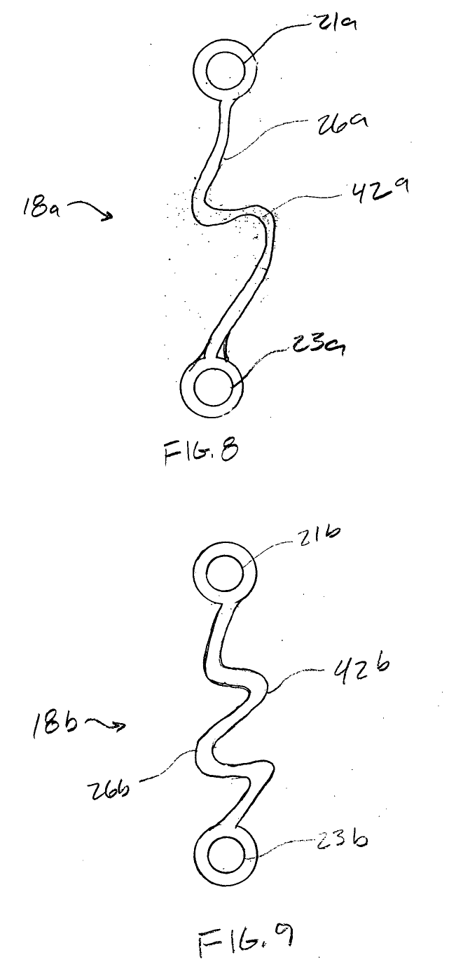

In one embodiment, the receivers 21, 23 may be configured such that the particular size of the receivers 21, 23 accommodate the fastener 24 and provides an excursion to allow the fastener 24 to move through the particular angle θ. Accordingly, the size of the receivers 21, 23 may be larger than a diameter of the head 30 of the fastener 24 to provide a clearance such that the fastener 24 may be allowed to move the particular angle θ, while being constrained from moving beyond the particular angle θ. In another embodiment, the receivers 21, 23 may include tapered or beveled openings 35, as shown most clearly in the portion of the implant 33 depicted in FIG. 7, to allow the fastener 24 to move the particular angle θ. It will be understood that the configuration of the fastener 24 may be compatible with the configuration of the receivers 21, 23 to enable constrained movement of the fasteners 24 to occur.

A discussion of the expander portion 26 will now be provided with reference to FIG. 3. It will be understood that the expander portion 26 may be provided in various different configurations as discussed below. One embodiment of the expander portion 26 may include a hollow sleeve 36 on one portion of the implant 18, and a rod 38 on an opposing portion of the implant 18. The rod 38 may be receivable in the sleeve 36 and moveable with respect to the sleeve 36. A spring 40 may also be located within the sleeve 36 to provide a damping or biasing force in the direction of arrow 41, to push the first end portion 20 away from the second end portion 22, and to absorb compressive forces exerted on the implant 18. It will be understood that the spring 40 may include a coiled member or the spring 40 may be formed in any other manner known to those skilled in the art. One embodiment of the spring 40 may be configured to abut with an end of the rod 38 and an interior end of the sleeve 36. The sleeve 36 may hold the spring 40 in place and provide support for the spring 40. It will also be understood that more than one spring 40 may be used in the implant 18, and that the springs may be attached and arranged in various different configurations within the scope of the present disclosure.

The strength and extension of the spring 40 may be selected based on the desired treatment. For example, it will be understood that a coiled spring 40 may reach its maximum force when the spring 40 is in a fully compressed position, whereas a leaf spring, as discussed more fully below, may reach its maximum force as the spring reaches its resting position. One embodiment of the spring 40 may be configured to provide 40-60 N of distraction force. However it will be understood that the spring 40 may be configured to provide any suitable force within the scope of the present disclosure.

It will be understood that the expander portion 26, in its various embodiments as disclosed herein, for example, may form a second excursion, in addition to the excursion provided by the first receiver 21 and/or the second receiver 23. Accordingly, the expander portion 26 may allow for additional movement between bone portions attached to the implant 18.

It will be understood that the spine in growing patients may grow at a rate of approximately 1 mm per year per segment, for example. Accordingly, a treatment requiring an implant 18 between two adjacent segments that may take two years to complete may initially require a spring 40 that allows 3-4 mm of movement, for example. However, a spring 40 allowing 4-6 mm of movement may be selected to compensate for the growth of the vertebrae during the treatment period. It will be understood that various different treatment periods may be used within the scope of the present disclosure. Moreover, the spring 40 may be sized to extend far enough to maintain a pushing force without becoming a tether and thereby providing a pulling force between the vertebrae or bone portions. Accordingly, one embodiment of the present disclosure may include a spring 40 that may be configured for providing a unidirectional distraction force without allowing a tensile force to be created in the spring 40. Also, the spring 40 may be sized to provide adequate force to prevent the rod 38 from bottoming out within the hollow sleeve 36.

One embodiment of the spring 40 may not be connected to the implant 18 on at least one end such that as the first end portion 20 of the implant 18 is separated a distance from the second end portion 22 of the implant 18, the spring 40 may not be tensioned to pull the first end portion 20 toward the second end portion 22. Other embodiments of the spring 40 may be attached to the implant to allow a tensile force in the spring 40 to be created, but the spring 40 may be sized so as to preclude a tensile force from being created in the spring 40 during normal operation. Other embodiments of the spring 40 may be configured to serve as a tether to provide a pulling force between the vertebrae.

One embodiment of the implant 18 of the present disclosure may also include a coating or jacket 39, as shown in dashed lines in FIG. 3 covering at least a portion of the implant 18. The jacket 39 may be formed of any suitable material, such as a polyethylene, silicon, or a di-block co-polymer such as polystyrene-polyethylene oxide (PS-PEO), for example, or other inert fabric material. The jacket 39 may be placed around the implant 18 to prevent soft tissue ingrowth, and to contain wear debris that may be generated by the implant 18. The jacket 39 may be fixed or removably joined with the implant 18 by sutures or any other suitable attachment mechanism known in the art.

It will be understood that the implant 18 and the fasteners 24 may be made of any suitable material known to those skilled in the art within the scope of the present disclosure. One embodiment of the implant 18 and the fasteners 24 may be made of, or include, a material that may be visible for inspection after being implanted into a body, such as a radiolucent material, for example.

Reference will now to made to FIG. 8 to describe an alternative embodiment of the present disclosure. As previously discussed, the presently disclosed embodiments of the disclosure illustrated herein are merely exemplary of the possible embodiments of the disclosure, including that illustrated in FIG. 8.

It will be appreciated that the alternative embodiment of the disclosure illustrated in FIG. 8 may contain many of the same structures represented in FIGS. 1-7 and only the new or different structures will be explained to most succinctly explain the features which come with the embodiments of the disclosure illustrated in FIG. 8.

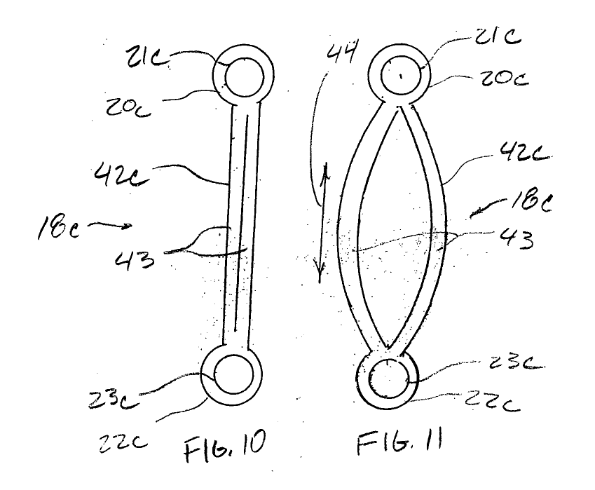

FIG. 8 illustrates a front view of an alternative embodiment implant, indicated at 18a. The implant 18a may include an expander portion 26a configured to provide a distraction force by a flat or leaf spring 42a. Accordingly, the implant 18a may be formed as a one piece unitary member including a first receiver 21a, second receiver 23a and expander portion 26a. The flat spring 42a may be configured to provide a low profile so as to reduce space required to accommodate the implant 18a. Moreover, it will be understood that further alternative embodiment implants 18b may be provided with any number of bends or loops, as depicted in the embodiment of the implant 18b shown in FIG. 9 having two bends or loops. Moreover, other embodiments may be formed with different shaped expander portions, such as angled linear segments, polygonal shapes, or any other suitable shape.

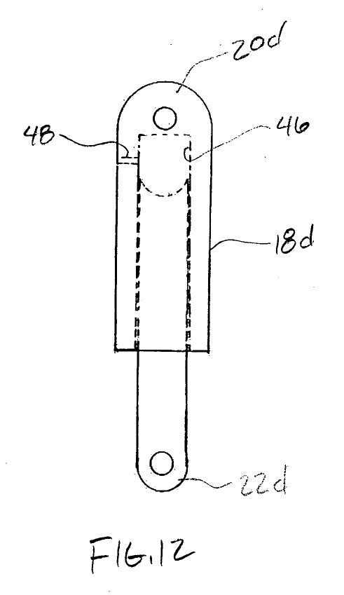

Referring now to FIGS. 10 and 11, an additional alternative embodiment of the present disclosure is shown. As previously discussed, the presently disclosed embodiments of the disclosure illustrated herein are merely exemplary of the possible embodiments of the disclosure, including that illustrated in FIGS. 10 and 11.

It will be appreciated that the alternative embodiment of the disclosure illustrated in FIGS. 10 and 11 may contain many of the same structures represented in FIGS. 1-9 and only the new or different structures will be explained to most succinctly explain the features which come with the embodiment of the disclosure illustrated in FIGS. 10 and 11.

FIG. 10 illustrates a front view of an alternative embodiment implant, indicated at 18c, in an extended position. The implant 18c may include a leaf spring 42c extending from the first end portion 20c to the second end portion 22c. The leaf spring 42c may include a plurality of legs 43 that may be configured to deflect laterally in a contracted position, as shown in FIG. 11. The resiliency of the legs 43 in the contracted position of FIG. 11 may create an expansion force to move the first receiver 21c in a direction away from the second receiver 23c as shown by the arrow 44. It will be understood that the spring 42c may be formed of any suitable material and may be configured to have a low profile to be received in a confined space.

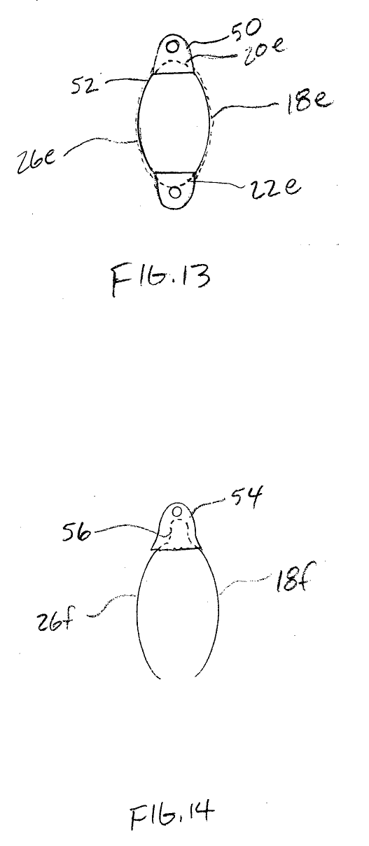

Referring now to FIG. 12 an additional alternative embodiment of the present disclosure is shown. As previously discussed, the presently disclosed embodiments of the disclosure illustrated herein are merely exemplary of the possible embodiments of the disclosure, including that illustrated in FIG. 12.

It will be appreciated that the alternative embodiment of the disclosure illustrated in FIG. 12 may contain many of the same structures represented in FIGS. 1-11 and only the new or different structures will be explained to most succinctly explain the features which come with the embodiments of the disclosure illustrated in FIG. 12.

FIG. 12 illustrates a front view of an alternative embodiment implant, indicated at 18d. The implant 18d may include a reservoir 46 for containing a material such as a hydrophilic gel. The hydrophilic gel may include a substance known in the art for imbibing fluid and expanding to thereby provide a distraction force to move the first end portion 20d of the implant 18d away from the second end portion 22 of the implant 18d. The implant 18d may include one or more ports 48 for connecting the reservoir 46 with surrounding body tissue fluids such that the hydrophilic gel may be configured to draw body fluids through the port 48 to the reservoir 46 to create the distraction force. It will be understood that the location, size and quantity of the ports 48 may vary in accordance with the principles of the present disclosure.

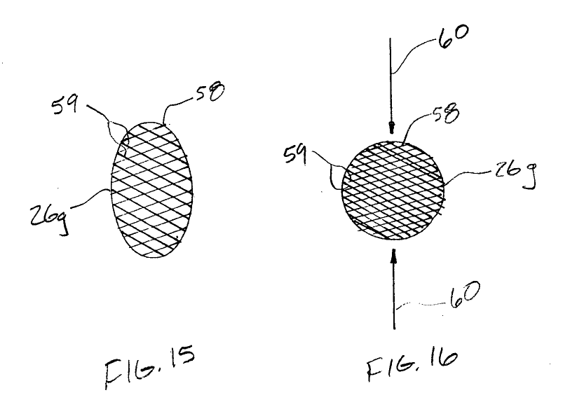

Reference will now be made to FIG. 13 to describe another alternative embodiment of the present disclosure. As previously discussed, the presently disclosed embodiments of the disclosure illustrated herein are merely exemplary of the possible embodiments of the disclosure, including that illustrated in FIG. 13. Moreover, the alternative embodiment of the disclosure illustrated in FIG. 13 may contain many of the same structures represented in FIGS. 1-12 and only the new or different structures will be explained to most succinctly explain the features which come with the embodiments of the disclosure illustrated in FIG. 13.

FIG. 13 illustrates a front view of another alternative embodiment implant, indicated at 18e. The implant 18e may include an expander portion 26e that may be formed of an elastic material, such as silicone rubber, for example. The expander portion 26e may be formed of a homogeneous material, or the expander portion 26e may be formed of multiple materials, such as expander portions 26e having reinforcing materials for providing additional strength or elasticity in particular areas of the expander portion 26e. It will be understood that the implant 18e may be formed in a compact configuration without any moving parts.

A cap 50 may be placed on one or both of the first end portion 20e and the second end portion 22e. The cap 50 may include a hollow space for receiving a portion of the expander portion 26e. One embodiment of the cap 50 may be formed in an approximate “trumpet flare” configuration. Moreover, the cap 50 may be formed of any suitable material, such as titanium, within the scope of the present disclosure. One embodiment of the implant 18e, may include a flexible sleeve 52, as shown in dashed lines in FIG. 13, for maintaining the cap 50 on the expander portion 26e, and/or for providing support for the expander portion 26e. The flexible sleeve 52 may be formed of any suitable material configured for deforming to maintain the cap 50 on the expander portion 26e as the expander portion 26e changes shape through expansion and contraction. For example, the flexible sleeve 52 may be formed of a tightly woven polyethylene material that may provide additional resistance to compression.

It will be understood that the expander portion 26e may have a somewhat egg or elliptical shape when the expander portion 26e is in a relaxed state. However, it will be understood that the expander portion 26e may have various other configurations, such as rounded or bulbous shapes, or any other suitable shape within the scope of the present disclosure. Some embodiments of the present disclosure may be formed without sharp corners which may create areas of increased stress. The expander portion 26e may be compressed toward a spherical or otherwise compacted configuration for being installed on one or more bones. The elastic properties of the expander portion 26e may cause the expander portion to create a distraction force as the expander portion 26e tries to move to its relaxed position. It will be understood that one embodiment of the implant 18e may be formed such that the fasteners 24e may be secured to the implant 18e without any allowable play, since the inherent elasticity of the expander portion 26e may accommodate movement of the fasteners 24e in torsion, side bending and flexion/extension.

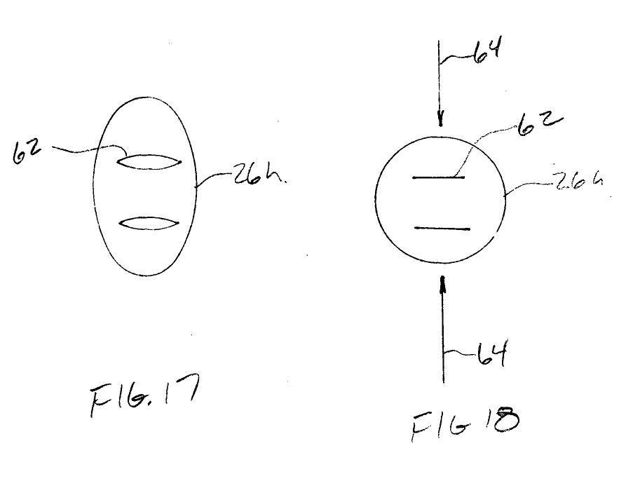

As shown in FIG. 14, an additional alternative embodiment implant 18f may be provided having a snap-fit cap 54. The expander portion 26f may include a snap-fit portion 56, such as a bulbous end, which may be configured to deflect or contract to snap-fit into a corresponding shape within the snap-fit cap 54. Accordingly, the cap 54 may be easily joined with the expander portion 26f. It will be understood that the snap fit portion 56 and snap-fit cap 54 may be formed in any suitable shape configured for providing a snap-fit connection within the scope of the present disclosure.

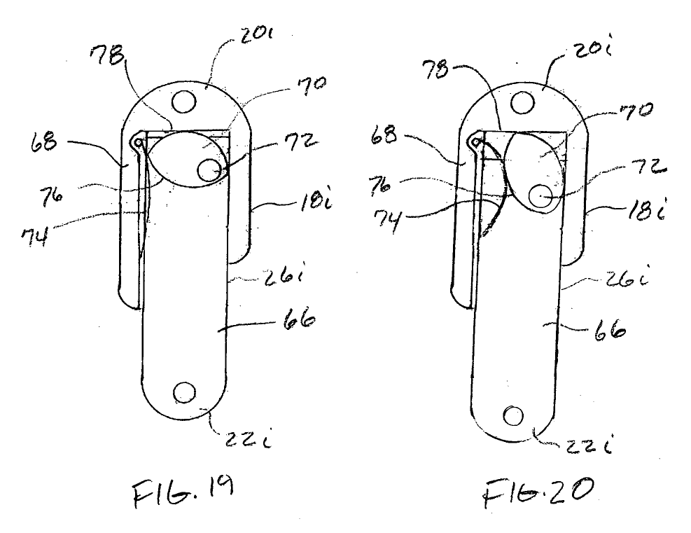

As shown in FIGS. 15 and 16, an expander portion 26g may be provided with a jacket 58 that may be woven or otherwise configured to assist in providing a distraction force. The jacket 58 may be formed of a mesh of strands 59 configured to deform or displace so as to re-enforce the expander portion 26g. As shown in FIG. 15, the expander portion 26g may form an elliptical or egg shaped member. When a compressive force, as indicated by arrows 60 in FIG. 16, is applied to the expander portion 26g, the expander portion 26g may compress to a rounded or spherical shape. The jacket 58 may also be deformed such that the strands 59 may be concentrated or closer together to increase support or resistance to deformation of the expander portion 26g.

It will be understood that the jacket 58 may be formed of any suitable material, and the arrangement of strands 59 may be formed in any suitable configuration. Moreover, other embodiments of the jacket 58 may be formed without strands such that the jacket 58 may be formed of a single piece or sheet member.

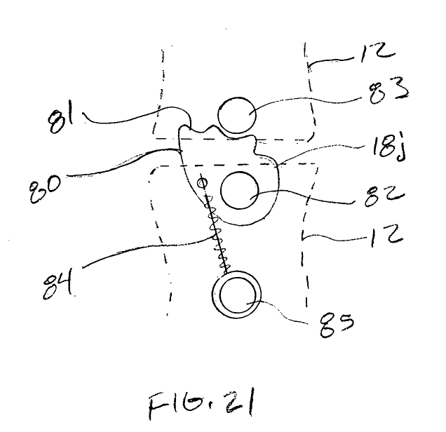

Referring now to FIGS. 17 and 18, an additional embodiment expander portion 26h is disclosed. Similar to some of the previously disclosed embodiments, the expander portion 26h may be elliptical or egg shaped in a relaxed condition. The expander portion 26h may include one or more fenestrations 62. The fenestrations 62 may be shaped and positioned to allow the expander portion 26h to compress more easily to a specified point, when a compressive force is applied to the expander portion 26h as indicated by arrows 64 in FIG. 18. Once the fenestrations 62 have collapsed to the point where a gap in the expander portion 26h has been eliminated, as shown in FIG. 18, the force required to further compress the expander portion 26h may increase. Accordingly, the distraction force provided by the expander portion 26h may not be linear or proportionate with respect to the displacement of the expander portion 26h.

It will be understood that the number, position and configuration of fenestrations 62 may be arranged to provide a desired distraction force for a particular situation. Additionally, other embodiments of the present disclosure may include an expander portion having a hollow interior or a solid interior. Moreover, a thickness of a sidewall forming the hollow expander portion, or the geometry of the expander portion, may be varied to provide a specified distraction force, either linearly or non-linearly, with respect to displacement of the expander portion.

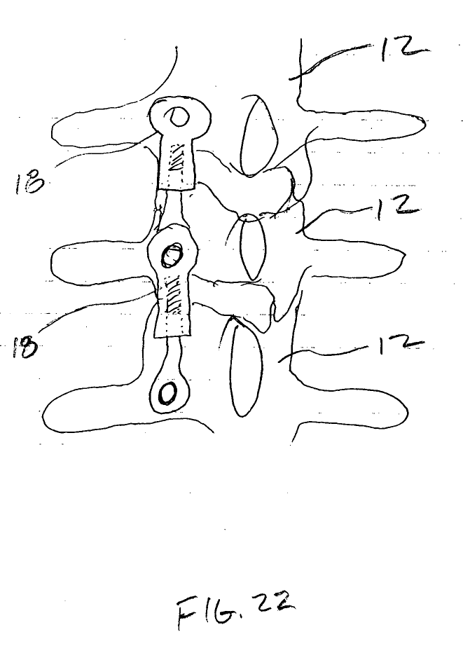

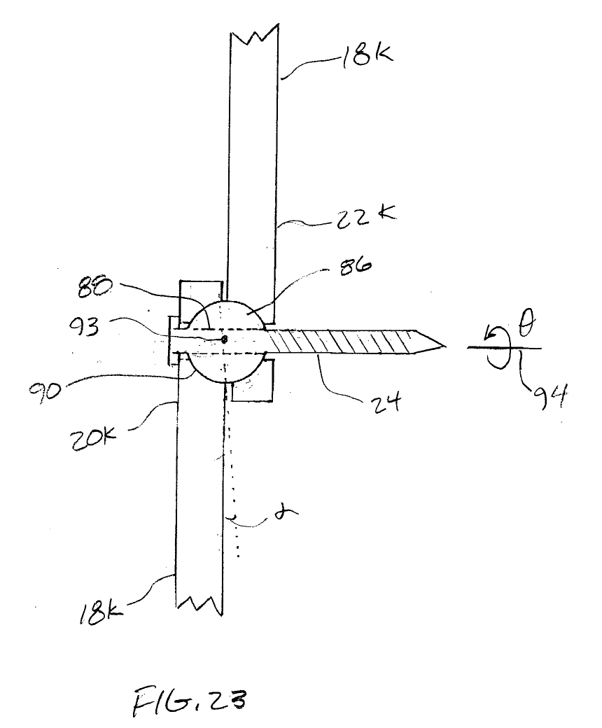

Referring now to FIGS. 19 and 20, an additional alternative embodiment of the present disclosure is shown. As previously discussed, the presently disclosed embodiments of the disclosure illustrated herein are merely exemplary of the possible embodiments of the disclosure, including that illustrated in FIGS. 19 and 20.

It will be appreciated that the alternative embodiment of the disclosure illustrated in FIGS. 19 and 20 may contain many of the same structures represented in FIGS. 1-18 and only the new or different structures will be explained to most succinctly explain the features which come with the embodiment of the disclosure illustrated in FIGS. 19 and 20.

FIG. 19 illustrates a front view of an alternative embodiment implant 18i in a contracted position. The implant 18i may include an expander portion 26i that may include a slider 66 and a housing 68. The housing 68 may define a space, slot, or groove for receiving the slider 66. The slider 66 may be movable with respect to the housing 68, as illustrated by the contracted view of the implant 18i shown in FIG. 19, as compared to an extended view of the implant 18i shown in FIG. 20.

The implant 18i may include a cam 70 that may be rotatably attached to the slider 66 by a pivot 72. The pivot 72 may be joined with the slider 66 such that the pivot 72 may be configured to move with the slider 66 as the slider 66 extends and contracts. A cam spring 74 may be attached to the housing 68 in any suitable manner known to those skilled in the art, for allowing the spring 74 to provide a bias force as it extends between a tensioned position, as shown in FIG. 19, and a relaxed position as shown in FIG. 20. The cam 70 may have a perimeter surface 76 that may contact the cam spring 74 on one side and an edge 78 of the housing 68 on another side. It will be understood that the cam spring 74 may press against the perimeter surface 76 of the cam 70 and cause the cam 70 to rotate about the pivot 72. The cam 70 may also contact the edge 78 of the housing 68 such that rotation of the cam 70 may thereby cause the slider 66 to move to the extended position shown in FIG. 20. It will be understood that various different spring and cam arrangements and configurations may be used to provide a distraction force within the scope of the present disclosure. Moreover, one embodiment of the implant 18i may be provided with stops formed in any manner known to those skilled in the art to limit the movement of the slider 66, or to prevent the slider 66 from separating from the housing 68.

Referring now to FIG. 21, a front view of another alternative embodiment implant 18j is shown. The implant 18j may include a ratchet 80 having one or more seats 81 formed in a perimeter thereof at different heights. The ratchet 80 may be rotatably attached to a bone or vertebra 12 through a first connector 82. The first connector 82 may include a fastener such as a screw, nail or pin, for example, for attaching the ratchet 80 to the vertebra 12.

A second connector 83 may also be connected to an opposing bone or vertebra 12. The second connector 83 may also be formed as a screw, nail, pin, or other such construct, for being received in a bone and being supported in a seat 81 of the ratchet 80. A biasing member 84 may be provided for providing a distraction force to the ratchet 80 by pushing the ratchet 80. The biasing member 84 may be rotatably attached to the vertebra 12 by a third connector 85. It will be understood that the biasing member 84 may be formed of any variety of spring known in the art for applying a pushing force on the ratchet 80 with respect to the third connector 85.

In use, the second connector 83 may be supported in a seat 81 of the ratchet 80. The biasing member 84 may push the ratchet 80 to an extended position to support the second connector in a higher seat 31. If an increased force is applied from the second connector 83 to the ratchet 80, the shape of the seat 81 may allow the ratchet 80 to rotate such that the second connector 83 may be supported on a lower seat 81. Other embodiments of the seats 81 may preclude the ratchet 80 from rotating to support the connector 83 on a lower seat 81. If pressure from the second connector 83 to the ratchet 80 is reduced, the distraction force provided by the biasing member 84 may cause the ratchet 80 to rotate in the opposite direction such that the second connector 83 may be supported in a higher seat 81. It will be understood that one embodiment of the implant 18j, as depicted in FIG. 20, may provide a distraction-force without constraining movement of the second connector 83 in a direction away from the ratchet 80, such that the implant 18j may not function as a tether to limit movement of one vertebra away from another.

Referring to FIG. 22, a break-away view is shown of a spine treated with a plurality of implants 18. It will be understood that the implants 18 may be arranged in an end to end configuration to span multiple segments. Accordingly, any number of implants 18 may be used to treat a bone or spine. Moreover, it will be understood that a single implant 18 may be sized to span multiple segments of a bone or vertebrae within the scope of the present disclosure. Thus, the implants 18 may be versatile such that the principles of the present disclosure may be used in various different configurations.

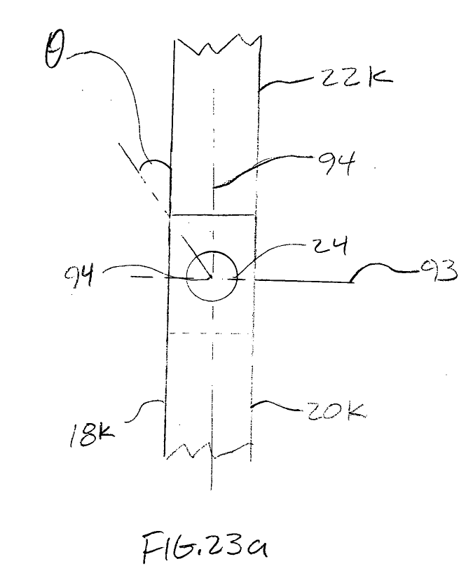

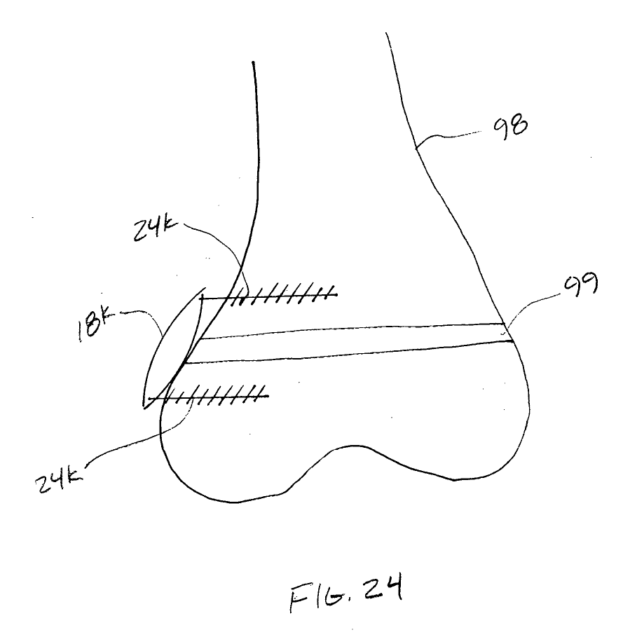

Referring to FIG. 23, a break-away side view is shown of a connection between a plurality of implants 18k, including a first end portion 20k of a first implant 18k, and a second end portion 22k of a second implant 18k. A joint 86 may be provided between the first end portion 20k of the first implant 18k, and the second end portion 22k of the second implant 18k. The joint 86 may include a passage 88 for receiving a fastener 24 to attach the implants 18k to a bone. One embodiment of the joint 86 may have a convex shape for being received in a corresponding concave shaped recess 90 formed in the implants 18k. The joint 86 may be moveable with respect to the first end portion 20k and the second end portion 22k. Accordingly, the first end portion 20k and the second end portion 22k may be allowed to move with respect to each other and with respect to the fastener 24. For example, the first end portion 20k and/or the second end portion 22k may be allowed to move at an angle α with respect to the fastener 24, about an axis 93 that may be perpendicular with respect to an axis 94 that may extend along a length of the fastener 24. Movement of the first end portion 20k and the second end portion 22k through the angle α may occur as the implants 18k extend or flex. Similarly, as shown most clearly in FIG. 23a, the first end portion 20k and/or the second end portion 22k may be allowed to rotate through an angle θ about the axis 94 through the fastener 24, with respect to the joint 86 in a different dimension than the angle α. Rotation through the angle θ may occur during side bending or rotation of the vertebrae or bone carrying the implants 18k. Accordingly, movement of the first end portion 20k and/or the second end portion 22k with respect to a fastener 24, as described herein, refers to at least movement about axis 93 and axis 94.

It will be understood that one embodiment of the joint 86 may be substantially spherical to be configured to allow movement of the first end portion 20k and the second end portion 22k through various different angular orientations or degrees of freedom within the scope of the present disclosure. Moreover, it will be understood that joint 86 and recesses 90 may have other configurations within the scope of the present disclosure.

A table showing allowable range of motion for distraction devices between thoracic (T) and lumbar (L) vertebral motion segments is presented below, as disclosed in Clinical Biomechanics of the Spine 2nd Ed, White A W III and Panjabi M M, J. B. Lippincott Co. Philadelphia, 1990. It will be understood that the table below shows representative ranges or values for various different movements.

Range of Motion (ROM) Allowance for Distraction Devices

| | | | |
|---|---|---|---|
| | Combined | One side lat. | One side axial |
| | Flex-Ext (°) | bending (°) | rotation (°) |
| Interspace | (α) | (θ) | (θ) |

| | | | | | | |
|---|---|---|---|---|---|---|
| T1-2 | 3-5 | 4 | 5 | 5 | 14 | 9 |
| T2-3 | 3-5 | 4 | 5-7 | 4 | 4-12 | 8 |
| T3-4 | 2-5 | 4 | 3-7 | 6 | 5-11 | 8 |
| T4-5 | 2-5 | 4 | 5-6 | 6 | 5-11 | 8 |
| T5-6 | 3-5 | 4 | 5-6 | 6 | 5-11 | 8 |
| T6-7 | 2-7 | 5 | 6 | 6 | 4-11 | 7 |
| T7-8 | 3-8 | 6 | 3-8 | 6 | 4-11 | 7 |
| T8-9 | 3-8 | 6 | 4-7 | 6 | 6-7 | 6 |
| T9-10 | 3-8 | 6 | 4-7 | 6 | 3-5 | 4 |
| T10-11 | 4-14 | 9 | 3-10 | 7 | 3-5 | 2 |
| T11-12 | 6-20 | 12 | 4-13 | 9 | 2-3 | 2 |
| T12-L1 | 6-20 | 12 | 5-10 | 8 | 2-3 | 2 |
| L1-2 | 5-16 | 12 | 3-8 | 6 | 1-3 | 2 |
| L2-3 | 8-18 | 14 | 3-10 | 6 | 1-3 | 2 |
| L3-4 | 6-17 | 15 | 4-12 | 8 | 1-3 | 2 |

The ROM may be described as the motion taking place between the stem of the fastener 24, such as a pedicle screw, and the implant 18. Also, an exemplary ROM for the implant 18 for the thoracic segments T1-T10 may be 8 degrees, and for the segments T10-L4 the ROM may be 11 degrees.

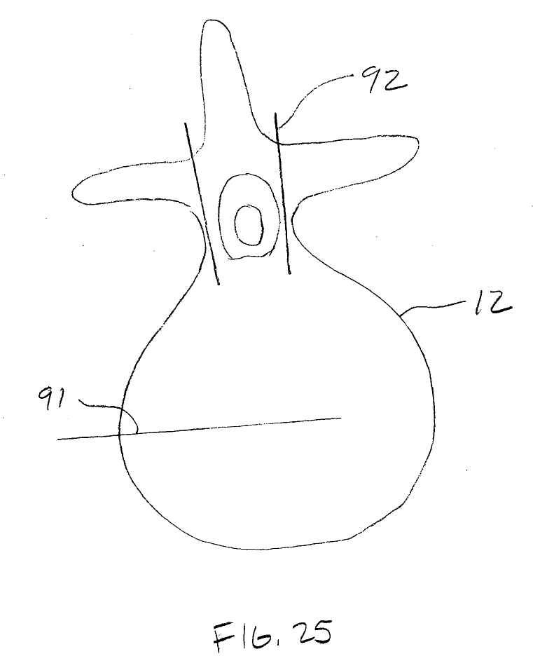

Referring now to FIG. 24, a side breakaway view is shown of a bone 98, such as a femur for example, being treated by an implant 18k in accordance with the principles of the present disclosure. The bone 98 may include a growth plate 99 which may benefit from a distraction force applied on opposing sides of the growth plate 99. Fasteners 24k, such as bone screws, may be installed on opposite sides of the growth plate 99 such that the implant 18k may be used to apply a distraction force between the fasteners 24k and thereby treat the bone 98. Accordingly, it will be understood that the principles of the present disclosure may be used to treat various different bones, including segments of a single bone, in addition to spinal deformities such as scoliosis. Moreover, the principles of the present disclosure may be utilized to treat other non-bone conditions.

In use, incisions may be made to access the vertebrae or other bone to be treated. When scoliosis is being treated by the implant 18, the vertebrae may be accessed and treated on the concave side of the spinal curve. It will be understood that the incisions may be made either on the anterior or the posterior side of a patient depending on the particular curvature to be treated. The vertebrae may be distracted initially as much as possible prior to installation of the implant 18. The fasteners 24 may be installed in the vertebrae at a particular position to allow adequate distraction force to be provided by the implant 18 without allowing the implant 18 to function as a tether. As shown in FIG. 25, which shows a schematic cross-sectional view of a vertebra 12, the fasteners 24 may be inserted using a less-invasive vertebral approach 91, or an open approach 92, depending on the particular treatment to be accomplished. It will also be understood that the fasteners 24 may be inserted thorascopically, or in any other suitable manner known to those skilled in the art. Moreover, the implant 18 may be sized and positioned to prevent the implant 18 from bottoming out, or being compressed to its limit under a compressive load. The implant 18 may be installed by placing the head 30 of the fasteners 24 in the receivers 21, 23, and installing the catches 34 in the grooves 32 to hold the head 30 of the fastener 24 within the receivers 21, 23. A jacket 39 may also be installed on the implant 18 to prevent soft tissue ingrowth and contain any wear debris that may be generated. The jacket 39 may be sutured to hold the jacket 39 in place.

It will be understood that in some situations, the implant 18 may be inserted through a posterior midline skin incision and then through a concave paramedian muscle splitting approach. However, it will be understood that any other suitable incision or approach may be utilized to install the implant 18 within the scope of the present disclosure

It will be understood that the implant 18 of the present disclosure may be provided as a dynamic implant that may allow for changes in dimension over time. In contrast to some prior art devices that provide a fixed amount of correction or treatment at the time of surgery, the principles of the present disclosure may be employed to allow for additional correction to occur over time due to changes in dimension of the device. It will be understood, however, that when the implant 18 of the present disclosure is utilized in younger patients, additional surgeries may be utilized to exchange the implant if desired. Additionally, the principles of the present disclosure may be utilized to form a non-fusion device. Moreover, the principles of the present disclosure may be utilized to provide a plurality of devices that allow for segmental load sharing over a length of a spine or bone.

It will be understood that the principles of the present disclosure may be used to treat idiopathic scoliosis, particularly when the patient has more than one year of growth remaining. Also, the present apparatus and methods may be used in cases where the patient has a flexible spine deformity which is unresponsive to orthotic treatment. Moreover, the apparatus and methods of the present disclosure may be used as an alternative to, or in combination with, growth rods.

It will be understood that the principles of the present disclosure may be used alone or in combination with various other types of treatment measures, such as growth stimulants, growth inhibitors, medications, or biological therapies, for example, to achieve a desired effect on the body being treated. Any variety of growth stimulants, growth inhibitors, medications, or biological therapies known to those skilled in the art may be used within the scope of the present disclosure. For example, the implant 18 and/or growth stimulants may be placed on the concave side 14 of the spine to enhance growth on the concave side 14 of the spine 10. Similarly, compression devices and/or growth inhibitors may be placed on the convex side 16 of the spine 10. Accordingly, treatments may be devised using a combination of mechanical devices and biological treatment measures to achieve the desired treatment of a spine or bone.

It will be appreciated that the structure and apparatus disclosed herein is merely exemplary of means for providing a distraction force, and it should be appreciated that any structure, apparatus or system for providing a distraction force which performs functions the same as, or equivalent to, those disclosed herein are intended to fall within the scope of a means for providing a distraction force, including those structures, apparatus or systems for providing a distraction force which are presently known, or which may become available in the future. Anything which functions the same as, or equivalently to, a means for providing a distraction force falls within the scope of this element.

It will be appreciated that the structure and apparatus disclosed herein is merely exemplary of means for joining with a fastener, and it should be appreciated that any structure, apparatus or system for joining with a fastener which performs functions the same as, or equivalent to, those disclosed herein are intended to fall within the scope of a means for joining with a fastener, including those structures, apparatus or systems for joining with a fastener which are presently known, or which may become available in the future. Anything which functions the same as, or equivalently to, a means for joining with a fastener falls within the scope of this element.

In accordance with the features and combinations described above, a useful method of distracting a first bone portion from a second bone portion may include:

(a) joining a first fastener with the first bone portion and a second fastener with the second bone portion on a concave side of a curve formed in the first bone portion and the second bone portion;

(b) joining an implant with the first fastener and the second fastener; (c) expanding the implant between the first bone portion and the second bone portion; and

(d) allowing angular movement of at least one of the first fastener and the second fastener with respect to the implant.

Those having ordinary skill in the relevant art will appreciate the advantages provide by the features of the present disclosure. For example, it is a feature of the present disclosure to provide a device for treating bones or spinal deformities such as scoliosis, which is simple in design and manufacture. Another feature of the present disclosure is to provide such a device for treating scoliosis which may provide a distraction force on a concave side of a spinal curve. It is a further feature of the present disclosure, in accordance with one aspect thereof, to provide a device for treating bones or scoliosis which may allow for confined movement of fasteners with respect to the device. It is another feature of the present disclosure to provide a device which may allow treatment of scoliosis while allowing movement of vertebrae with respect to each other and maintaining a distraction force as a patient grows. It is an additional feature of the present disclosure to provide a device for treating scoliosis without fusing vertebrae. It is a further feature of the present disclosure to provide a device for treating bones which device may be implanted in a body with minimal trauma to the body such that the device may be minimally invasive. It is yet an additional feature of the present disclosure to provide a device for treating bones which may allow for changes in dimension over time.

In the foregoing Detailed Description, various features of the present disclosure are grouped together in a single embodiment for the purpose of streamlining the disclosure. This method of disclosure is not to be interpreted as reflecting an intention that the claimed disclosure requires more features than are expressly recited in each claim. Rather, as the following claims reflect, inventive aspects lie in less than all features of a single foregoing disclosed embodiment. Thus, the following claims are hereby incorporated into this Detailed Description of the Disclosure by this reference, with each claim standing on its own as a separate embodiment of the present disclosure.

It is to be understood that the above-described arrangements are only illustrative of the application of the principles of the present disclosure. Numerous modifications and alternative arrangements may be devised by those skilled in the art without departing from the spirit and scope of the present disclosure and the appended claims are intended to cover such modifications and arrangements. Thus, while the present disclosure has been shown in the drawings and described above with particularity and detail, it will be apparent to those of ordinary skill in the art that numerous modifications, including, but not limited to, variations in size, materials, shape, form, function and manner of operation, assembly and use may be made without departing from the principles and concepts set forth herein.

What is claimed is:

1. A device for distracting one or more bones, said device comprising:
a first end portion having a first receiver for receiving a first fastener to attach said first end portion to a first bone portion;
a second end portion having a second receiver for receiving a second fastener to attach said second end portion to a second bone portion;
an expander portion between said first end portion and said second end portion;
wherein at least one of said first receiver and said second receiver defines a dynamic connection which allows constrained movement of up to 20 degrees of at least one of said first end portion and said second end portion with respect to at least one of said first fastener and said second fastener.

2. The device of claim 1, wherein said expander portion comprises a sleeve joined to said first end portion and a rod joined to said second end portion, said rod being movably received in said sleeve.

3. The device of claim 2, further comprising a spring in said sleeve to bias said second end portion away from said first end portion.

4. The device of claim 1, further comprising a jacket surrounding at least a portion of said expander portion for preventing soft tissue ingrowth.

5. The device of claim 1, wherein said first receiver and said second receiver each comprise an opening configured for receiving said first fastener and said second fastener, respectively.

6. The device of claim 5, wherein said openings are sized to allow a predetermined amount of movement of said first fastener and said second fastener within said openings.

7. The device of claim 5, wherein said openings are beveled to allow a predetermined amount of movement of said first fastener and said second fastener within said openings.

8. The device of claim 1, wherein at least one of said first receiver and said second receiver are configured for receiving a movable rounded member for allowing movement of one of said first fastener and said second fastener.

9. The device of claim 8, wherein at least one of said first receiver and said second receiver comprises a stop for limiting movement of one of said first fastener and said second fastener.

10. The device of claim 1, wherein at least one of said first receiver and said second receiver is configured to move with respect to one of said first fastener and said second fastener through an angle of up to 8 degrees about an axis perpendicular to an axis of said one of said first fastener and said second fastener.

11. The device of claim 1, wherein at least one of said first receiver and said second receiver is configured to move with respect to one of said first fastener and said second fastener about an axis through a length of one of said first fastener and said second fastener.

12. The device of claim 11, wherein said expander portion comprises a flat spring comprising a plurality of bends.

13. The device of claim 1, wherein said expander portion, said first end portion and said second end portion are formed as a one piece unitary member.

14. The device of claim 1, wherein said expander portion comprises a spring having a plurality of legs.

15. The device of claim 1, wherein said expander portion comprises a reservoir containing a hydrophilic gel.

16. The device of claim 15, further comprising a port for connecting said reservoir with surrounding fluids.

17. The device of claim 1, wherein said expander portion comprises an elastic material that is deformable to provide a distraction force.

18. The device of claim 17, further comprising a mesh of strands surrounding said elastic material.

19. The device of claim 17, further comprising one or more fenestrations in said elastic material.

20. The device of claim 17, wherein said first end portion and said second end portion each comprise a cap for covering at least a portion of said elastic material.

21. The device of claim 20, wherein said elastic material comprises a snap-fit portion for attaching to a cap with a snap-fit.

22. The device of claim 20, further comprising a flexible sleeve for joining said caps with said elastic material.

23. The device of claim 1, wherein said expander portion comprises a housing receiving a slider.

24. The device of claim 23, further comprising a cam rotatably joined to said slider.

25. The device of claim 24, further comprising a cam spring for biasing said cam to rotate and thereby cause said slider to move to an extended position.

26. A device for distracting one or more bones, said device comprising:
a first end portion having a first receiver for receiving a first fastener to attach said first end portion to a first bone portion;
a second end portion having a second receiver for receiving a second fastener to attach said second end portion to a second bone portion; and
an expander portion between said first end portion and said second end portion;
wherein said first end portion, said second end portion and said expander portion are collectively formed of a one piece unitary construction; and
wherein at least one of said first receiver and said second receiver is configured to allow a predetermined amount of movement of one of said first end portion and said second end portion relative to one of said first fastener and said second fastener, respectively, when said first fastener or said second fastener is held in a substantially fixed orientation.

27. The device of claim 26, further comprising a jacket surrounding at least a portion of said expander portion.

28. The device of claim 26, wherein said first receiver and said second receiver each comprise an opening configured for receiving said first fastener and said second fastener, respectively.

29. The device of claim 28, wherein said openings are sized to allow a predetermined amount of movement of said first fastener and said second fastener within said openings.

30. The device of claim 28, wherein said openings are beveled to allow a predetermined amount of movement of said first fastener and said second fastener within said openings.

31. The device of claim 26, wherein at least one of said first receiver and said second receiver is configured to allow at least one of said first fastener and said second fastener to move through an angle of up to 8 degrees.

32. The device of claim 26, wherein said expander portion comprises a flat spring.

33. The device of claim 32, wherein said flat spring comprises a plurality of bends.

34. The device of claim 26, wherein said expander portion comprises a spring having a plurality of legs.

35. A device for distracting one or more bones, said device comprising:
a first end portion having a first receiver defining an opening for receiving a first fastener to attach said first end portion to a first bone portion;
a second end portion having a second receiver defining an opening for receiving a second fastener to attach said second end portion to a second bone portion;
an expander portion between said first end portion and said second end portion, said expander portion providing a unidirectional distraction force;
wherein at least one of said first receiver and said second receiver is configured to allow a predetermined amount of movement of one of said first end portion and said second end portion relative to one of said first fastener and said second fastener, respectively, when said first fastener or said second fastener is held in a substantially fixed orientation.

36. The device of claim 35, wherein said expander portion comprises a sleeve joined to said first end portion and a rod joined to said second end portion, said rod being movably received in said sleeve.

37. The device of claim 36, further comprising a spring in said sleeve to provide said unidirectional distraction force.

38. The device of claim 35, further comprising a jacket surrounding at least a portion of said expander portion.

39. The device of claim 35, wherein said openings are sized to allow a predetermined amount of movement of said first fastener and said second fastener within said openings.

40. The device of claim 35, wherein said openings are beveled to allow a predetermined amount of movement of said first fastener and said second fastener within said openings.

41. The device of claim 35, wherein at least one of said first receiver and said second receiver comprises a movable rounded member for allowing movement of one of said first fastener and said second fastener.

42. The device of claim 35, wherein at least one of said first receiver and said second receiver comprises a stop for limiting movement of one of said first fastener and said second fastener.

43. The device of claim 35, wherein at least one of said first receiver and said second receiver is configured to allow at least one of said first fastener and said second fastener to move through an angle of up to 8 degrees.

44. A device for distracting one or more bones, said device comprising:
a first end portion having a first opening;
a second end portion having a second opening; and
means for providing a distraction force between said first end portion and said second end portion;
wherein at least one of said first end portion and said second end portion comprises means for joining with a fastener such that said at least one of said first end portion and said second end portion is configured to allow a predetermined amount of movement of said at least one of said first end portion and said second end portion relative to said fastener when said fastener is held in a substantially fixed orientation.

45. The device of claim 44, wherein said means for providing a distraction force comprises a hollow sleeve joined with said first end portion and a rod joined with said second end portion, said rod being receivable in said sleeve.

46. The device of claim 45, wherein a coiled spring is disposed in said hollow sleeve.

47. The device of claim 44, wherein a reservoir is formed in said hollow sleeve for receiving a hydrophilic gel.

48. The device of claim 44, wherein said means for providing a distraction force comprises a leaf spring.

49. The device of claim 44, wherein said means for providing a distraction force comprises a cam.

50. The device of claim 44, wherein said means for providing a distraction force comprises an elastic material.

51. The device of claim 44, wherein said means for joining with a fastener comprises a beveled opening.

52. The device of claim 44, wherein said means for joining with a fastener comprises a rounded joint.

53. The device of claim 44, wherein said means for joining with a fastener comprises an opening having a predetermined size with respect to said fastener for allowing said predetermined amount of movement.

54. The device of claim 44, wherein said predetermined amount of movement of said fastener with respect to said device is less than 20 degrees.

55. A method for distracting a first bone portion from a second bone portion, said method comprising:
(a) joining a first fastener with said first bone portion and a second fastener with said second bone portion on a concave side of a curve formed in said first bone portion and said second bone portion;
(b) joining an implant with said first fastener and said second fastener;
(c) expanding said implant between said first bone portion and said second bone portion; and
(d) allowing angular movement of at least one of said first fastener and said second fastener with respect to said implant.

56. The method of claim 55, further comprising joining a plurality of said implants in an end to end configuration.

57. The method of claim 55, further comprising allowing a dimension of said implant to change to accommodate physiological growth.

58. The method of claim 55, further comprising providing a damping force with said implant.

59. The method of claim 55, further comprising installing a jacket on said implant.

60. The method of claim 55, further comprising providing a biological therapy to at least one of said first bone portion and said second bone portion.

61. The method of claim 55, further comprising preventing said implant from forming a tether that pulls said first bone portion toward said second bone portion.

62. The method of claim 55, further comprising preventing said angular movement of said at least one of said first fastener and said second fastener with respect to said implant beyond 8 degrees.

63. A method for distracting a first bone portion from a second bone portion, said method comprising:
(a) providing an implant having a first end portion and a second end portion receivable in said first end portion;
(b) joining said first end portion with said first bone portion on a concave side of a curve formed in said first bone portion and said second bone portion;
(c) joining said second end portion with said second bone portion on said concave side of said curve; and
(d) providing a biasing force with said implant between said first bone portion and said second bone portion.

64. The method of claim 63, wherein joining said first end portion with said first bone portion comprises joining a first fastener with said first bone portion.

65. The method of claim 64, wherein joining said second end portion with said second bone portion comprises joining a second fastener with said second bone portion.

66. The method of claim 63, further comprising allowing angular movement of at least one of said first fastener and said second fastener with respect to said implant.

67. The method of claim 66, further comprising preventing said angular movement of said at least one of said first fastener and said second fastener with respect to said implant beyond 8 degrees.

68. The method of claim 63, further comprising joining a plurality of said implants in an end to end configuration.

69. The method of claim 63, further comprising allowing a dimension of said implant to change to accommodate physiological growth.

70. The method of claim 63, further comprising providing a damping force with said implant.

71. The method of claim 63, further comprising installing a jacket on said implant.

72. The method of claim 63, further comprising providing a biological therapy to at least one of said first bone portion and said second bone portion.

73. The method of claim 63, further comprising preventing said implant from forming a tether that pulls said first bone portion toward said second bone portion.

74. A method for distracting a first bone portion from a second bone portion, said method comprising:
(a) joining an implant with said first bone portion and said second bone portion on a concave side of a curve formed in said first bone portion and said second bone portion;
(b) expanding said implant between said first bone portion and said second bone portion; and
(c) preventing said implant from forming a tether that pulls said first bone portion toward said second bone portion.

75. The method of claim 74, wherein joining said implant with said first bone portion comprises joining a first fastener with said first bone portion.

76. The method of claim 75, wherein joining said implant with said second bone portion comprises joining a second fastener with said second bone portion.

77. The method of claim 74, further comprising allowing angular movement of at least one of said first fastener and said second fastener with respect to said implant.

78. The method of claim 77, further comprising preventing said angular movement of said at least one of said first fastener and said second fastener with respect to said implant beyond 8 degrees.

79. The method of claim 74, further comprising joining a plurality of said implants in an end to end configuration.

80. The method of claim 74, further comprising allowing a dimension of said implant to change to accommodate physiological growth.

81. The method of claim 74, further comprising providing a damping force with said implant.

82. The method of claim 74, further comprising installing a jacket on said implant.

83. The method of claim 74, further comprising providing a biological therapy to at least one of said first bone portion and said second bone portion.

84. A method for distracting a first bone portion from a second bone portion, said method comprising:
(a) joining an implant with said first bone portion and said second bone portion on a concave side of a curve formed in said first bone portion and said second bone portion;
(b) expanding said implant between said first bone portion and said second bone portion to provide a distraction force; and
(c) allowing a dimension of said implant to change to accommodate physiological growth.

85. The method of claim 84, wherein joining said implant with said first bone portion comprises joining a first fastener with said first bone portion.

86. The method of claim 85, wherein joining said implant with said second bone portion comprises joining a second fastener with said second bone portion.

87. The method of claim 84, further comprising allowing angular movement of at least one of said first fastener and said second fastener with respect to said implant.

88. The method of claim 87, further comprising preventing said angular movement of said at least one of said first fastener and said second fastener with respect to said implant beyond 8 degrees.

89. The method of claim 84, further comprising joining a plurality of said implants in an end to end configuration.

90. The method of claim 84, further comprising providing a damping force with said implant.

91. The method of claim 84, further comprising installing a jacket on said implant.

92. The method of claim 84, further comprising providing a biological therapy to at least one of said first bone portion and said second bone portion.
